(19) United States
(12) Patent Application Publication
(10) Pub. No.: US 2022/0168473 A1
(43) Pub. Date: Jun. 2, 2022

(54) IMPLANT WITH INTRINSIC ANTIMICROBIAL EFFICACY, AND METHOD FOR THE PRODUCTION THEREOF

(71) Applicant: Karl Leibinger Medizintechnik GmbH & Co. KG, Mühlheim (DE)

(72) Inventors: Adem AKSU, Villingen--Schwenningen (DE); Frank REINAUER, Emmingen-Liptingen (DE); Tobias WOLFRAM, Dreieich (DE)

(21) Appl. No.: 17/441,404

(22) PCT Filed: Jan. 28, 2020
(86) PCT No.: PCT/EP2020/052068

§ 371 Date: Sep. 21, 2021

(30) Foreign Application Priority Data
Mar. 29, 2019 (DE) 10 2019 108 327.3

(51) Int. Cl. A61L 27/54; A61L 27/44; A61L 27/42; A61L 27/56; A61F 2/02

(52) U.S. Cl. CPC A61L 27/54; A61L 27/446; A61L 27/427; A61F 2240/001; A61F 2/02; A61L 2300/404; A61L 2300/104; A61L 27/56

(57) ABSTRACT

The invention relates to an implant (1) with antimicrobial activity, comprising an implant mixture (IM) which has a base granular material (2) formed from a raw material mixture of biocompatible polymers and/or a ceramic granular material, the implant mixture (IM) also comprising at least one type of metal (3) in particle form which is suitable for releasing ions, the metal particles (3) being present in the form of silver particles and/or copper particles. The metal particles (3) are distributed in the volume of the implant (1). The invention also relates to a method for producing an implant (1) of said type.

The invention relates to an implant with antimicrobial activity comprising an implant mixture having a base granular material made of a raw material mixture of biocompatible polymers, for example UHMW-PE, polyurethane, HDPE or LDPE, PPSU, PP, PEEK and/or a ceramic granular material, such as calcium carbonate, wherein the implant mixture further comprises at least one kind of particulate metal suitable for releasing ions, wherein the metal particles are provided in the form of silver particles and/or copper particles. Furthermore, the invention relates to a method for manufacturing such an implant.

An implant is understood to be a medical device that is foreign to the body and is present in a human or animal body, in particular for a defined period of time.

Implants with antimicrobial activity/efficacy/effect reduce the ability of microorganisms to multiply and/or their infectivity and/or kill or inactivate them in order to suppress inflammations/diseases in the patient. Such microorganisms can be classified as bacteria, fungi, yeasts and viruses.

A biocompatible implant is an implant that has no negative influence on the metabolism in the human/animal body and, for example, does not cause any rejection reactions of the body for this implant. Thus, a (partially) biocompatible implant may remain in the patient's body for a long period of time.

However, it is known from the past that porous implants made of materials such as biocompatible polymers may cause infections and associated inflammatory reactions when implants are inserted into a (patient's) body. The subsequently occurring immunological reactions against bacteria that were brought in during surgery or were already present in the patient's tissue due to previous infections lead to a loss of function of the implant and furthermore to considerable impairments of the patient. Often these implants have to be removed because antibiotic treatment is ineffective due to biofilm formation on the implant and the implant has good conditions for bacterial adhesion due to the porosity which possibly exists.

In order to prevent this bacterial adhesion, implants may be provided with a coating acting in an antimicrobial manner. Often, these coatings are not stable and are only effective for a short period of time. In addition, coatings pose a technical problem for implants that have high or low porosity or partial porosity. Often the coatings are incompletely applied or have different layer thicknesses with insufficient activity. In addition to traditional antibiotics, various peptides with antimicrobial properties are also used to produce coated implants. Alternatively, certain metallic ions, such as silver ions or copper ions, are also used to produce an antimicrobially active coating.

Existing solutions with biopolymers relate, inter alia, to water-based coatings. Here, antibiotic-containing solutions or peptide solutions are applied to the implant surface, for example, in a peat coating process. The antimicrobial substance then acts by diffusion in the tissue. However, most coatings have only a short period of activity (less than six months) because the substances themselves are thermally unstable or the reservoir of these substances is exhausted to the maximum after this time.

Another way to make the implant antimicrobially effective is known from/used in drug delivery. Drug delivery refers to methods and systems for transporting a pharmaceutical component into a patient's body in order to safely achieve a desired therapeutic effect via the corresponding antimicrobial substance. In drug delivery, resorbable (carrier) materials (materials/substances that a living being can absorb) release pharmacological substances (substances that interact with a patient's body). These substances are distributed by diffusion and do not act directly on the implant/not in the direct environment of the implant, but only act in the distal (patient) tissue on specific target cells.

For example, EP 2 382 960 A1 describes an implant with a coating which releases silver ions in the human body and thus has an antimicrobial effect. A first surface portion of the coating is formed by an anode material. A second surface portion of the coating is formed by a cathode material. The cathode material is higher in the electrochemical series than the anode material, and the cathode material and the anode material are connected to each other in an electrically conductive manner.

Furthermore, an implant with long-term antibiotic effect is known from EP 1 513 563 B1, which in particular is a vascular prosthesis, with a basic structure determining the shape of the implant made of essentially non-resorbable or only slowly resorbable polymer material and a coating made of a resorbable material. There is a layer of metallic silver on the polymeric material and under the coating.

In addition, a method for producing an anti-infective coating on implants containing or consisting of titanium is disclosed in EP 2 204 199 B1. The method uses the following steps: formation of a porous oxide layer by anodic oxidation in an alkaline solution in such a way that the conductivity in the pores enables galvanic deposition, galvanic deposition of a metal with anti-infective properties, and solidification of the metal-containing oxide layer by blasting.

Furthermore, EP 3 424 877 A discloses an implant having an antimicrobial property which is generated thereby that silver particles are incorporated into calcium phosphate particles (bio ceramic), which is then melted and afterwards calcined such that the silver particles are spread all over the entire volume of the implant. For the production of the implant, calcium phosphate particles are mixed with the silver particles. Via centrifugation and drying dry mixed particles are obtained which are then pressed under heat.

WO 95/20878 A D2 discloses against the background of medical application a method for producing antimicrobial plastics using metal particles wherein these metal particles are embedded into the plastic in form of discrete particles. For this purpose, granulate-shaped plastic particles are coated with the metal particles, are then crushed/fused and afterwards brought into their desired shape.

In addition, WO 02/17984 A describes an antimicrobial material for being implanted into bones which is constructed such that metal particles are finely distributed within a matrix material which is a polymer.

Against this background, it is the object of the present invention to solve or at least reduce the problems of the prior art and, in particular, to provide an implant which can be manufactured at low cost and which acts reliably and safely against microorganisms.

This object is solved by the present invention in that the metal particles are distributed, preferably uniformly, in the volume of the implant/implant mixture. This means that the antimicrobial efficacy is distributed over the (entire) volume of the implant and is thus provided in a structurally intrinsic way in the implant, so that the antibacterial activity is a property of the implant itself. For this purpose, the implant mixture, in addition to the silver particles and/or copper particles, is interspersed with further metal particles in the form of magnesium particles and/or iron particles, which are highly pure and elemental as well as biodegradable metals.

The advantage of the implant designed in this way is that implants with metal particles distributed over the volume, which create the antimicrobial properties of the implant, have a significantly longer and more reliable antimicrobial effect than implants with an antimicrobial coating. In addition, the antimicrobial effect in the implant according to the invention takes place in its direct environment, so that any microorganisms present in the implant cannot spread in the patient's body.

Advantageous embodiments are the subject matter of the dependent claims and are explained in more detail below.

The implant according to the invention provides that the implant mixture, preferably in addition to the silver particles and/or copper particles, is interspersed with further metal particles in the form of magnesium particles and/or iron particles. These magnesium particles and/or iron particles, like the silver particles and/or copper particles, have an antimicrobial effect and thus increase the antimicrobial activity of the implant. Mixing the silver particles and/or copper particles with magnesium particles and/or iron particles leads to better tissue ingrowth behavior in the patient's body.

Furthermore, the implant is provided in such a way that the metal particles are highly pure and elemental as well as biodegradable metals. Such biodegradable metals are metals that are chemically or biologically degradable and are no longer present in the implant or in the patient's body after complete degradation.

Furthermore, it is conceivable that the distribution, density, quantity and/or concentration of the metal particles in the implant mixture is such that the antimicrobial activity of the implant is forced to occur/acts in its direct environment, i.e. directly on the surface of the implant, on the implant itself and to the maximum in an environment with a distance of 1-2 μm from the surface of the implant. In the case that the antimicrobial activity acts directly on the implant, it is prevented that microorganisms starting from the implant can spread in the surrounding tissue of the patient and thus possibly cause inflammations/diseases in the patient's body.

Advantageously, the implant can be designed in such a way that the silver particles have a grain size in the range of 1-200 μm, in particular 20 to 50 μm, the copper particles have a grain size in the range of 1-100 μm, in particular 10-30 μm, and the magnesium particles and iron particles have a grain size in the range of 1-200 μm. In this size range, the particles are particularly easy to introduce into the implant mixture.

It is also conceivable that the implant is porous and preferably such a distribution, density, quantity and/or concentration of the metal particles in the implant mixture is selected that the antimicrobial activity of the porous implant acts/is forced to occur on the pore surface. The pore surface is defined as the surface of all pores in the implant and is thus larger than the implant surface.

Furthermore, the implant can be designed in such a way that it is solid and preferably such a distribution, density, quantity and/or concentration of the metal particles in the implant mixture is selected that the antimicrobial activity of the solid implant acts/is forced to occur on the implant surface. In the case where the implant is solid, the antimicrobial activity acts only on the implant surface and thus on a smaller surface than in the case where the implant is porous.

It is also advantageous if the implant is produced with patient-specific shape and material properties. A patient-specific implant is an implant that is adapted/tailored to the individual anatomy of a patient.

It is also conceivable that the implant is produced by compression molding, milling, laser sintering, or injection molding. These are particularly effective production methods for producing an implant.

Furthermore, the object of the present invention is solved by a method for producing an implant with intrinsic antimicrobial activity. The implant has the implant mixture according to the invention defined above.

It is convenient if the method for producing the implant comprises the following steps, which are preferably carried out successively and in the following sequence:

a) mixing of the raw materials for producing the base granular material (, then)

b) mixing or blasting the base granular material with the silver particles and/or copper particles, optionally in combination with magnesium particles and/or iron particles, in a defined ratio, wherein the implant mixture is formed, and (then)

c) pressing the implant mixture for producing a material block which is preferably crushed into chunks in subsequent steps, for example by machining or grinding, and wherein these chunks are subsequently formed into the (desired) final implant shape.

In other words, the present invention relates to a method for producing an antimicrobial granular material as a starting material for producing differently dimensioned implants with different porosities and partial resorbability. The starting material (UHMW-PE, HDPE, PP, polyurethane, LDPE, magnesium particles, PPSU) may be provided as granular material or as powder.

Furthermore, in other words, the invention relates to an implant (permanent implant or partially resorbable implant) with intrinsic antimicrobial effect, which is independent of the porosity and the geometric design of the porosity and/or pores. The antibacterial substance is not applied to the implant as a coating, but is part of the particulate base material of the implant.

The production of the solid, porous, highly porous or geometrically complex implants with their antimicrobial effect is based on the addition of silver particles or copper particles, which release ions over time. Highly pure, microporous silver is used to treat inflammatory complications. The antibacterial activity of an implant can also occur partially during resorption of implant parts.

By mixing biodegradable metallic particles of magnesium or iron alloys together with silver particles or copper particles added to the base granular material consisting of polymers or ceramic particles and/or thermal particles of mixtures of these base materials, a better tissue ingrowth behavior is achieved. Here, the fully/partially porous and three-dimensional implant exhibits antimicrobial activity regardless of whether the surface is initially accessible (opened pores) or not (closed pores).

The implant-raw materials are produced and mixed in a solvent-free manner. The base granular material/powder is activated by mixing in defined ratios with silver material or copper particles. The base granular material/powder can alternatively be combined with silver or copper by blasting. The combination of magnesium particles or iron particles together with silver particles or copper particles in a polymeric or ceramic background matrix (base granular material) depends on the thermal or mechanical manufacturing process. The implant mixture is then pressed and subsequently crushed/ground into granular material.

Thus, a compression-molded, milled, laser-sintered or injected implant made of biocompatible polymers or ceramic granular materials with an antimicrobial effect is obtained by adding (preferably nanoparticulate) high-purity elemental silver particles and/or copper particles. The antimicrobial activity of porous implants is limited to the effect of the pore surface (external and internal). In contrast, the antimicrobial activity of solid implants is effective only on the implant surface. The antimicrobial activity is cell-compatible and cell-physiologically harmless, since the concentration of metal particles acts only in the immediate vicinity of the implant due to the technical implant design. A highly porous implant maintains the antimicrobial activity without closing the pores.

Other materials that may have implants with antimicrobial activity include PEEK, PPSU with included additives such as hydroxylapatite (HA), calcium carbonate (CaCO₃), strontium (Sr), α- or β-tricalcium phosphate (α- or β-TCP), bioglass particles/particles of bioactive glass, a polyester material such as PDLLA, PLGA, PLA, PGA, chitosan fibers or chitosan particles. A porous implant achieves better ingrowth behavior into the patient's body compared to a non-porous/solid implant, without limiting/losing the antimicrobial effect of the implant due to porosity. The strength of the implant according to the invention can be increased by blasting, spraying, mixing, granulating or pressing.

The following describes in detail an embodiment of the implant according to the invention and the method of producing the implant with reference to the accompanying drawings.

The following is shown:

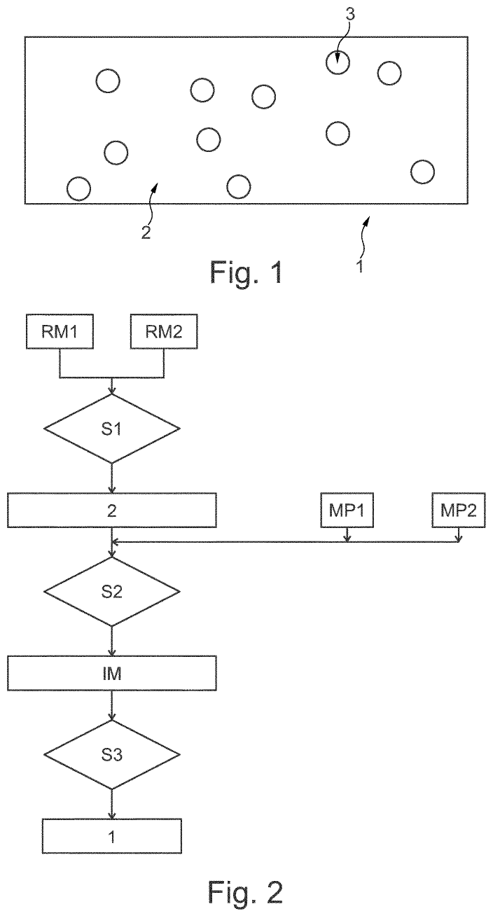

FIG. 1 shows a schematic cross-sectional view of an implant;

FIG. 2 shows a flowchart illustrating the steps involved in the production of the implant.

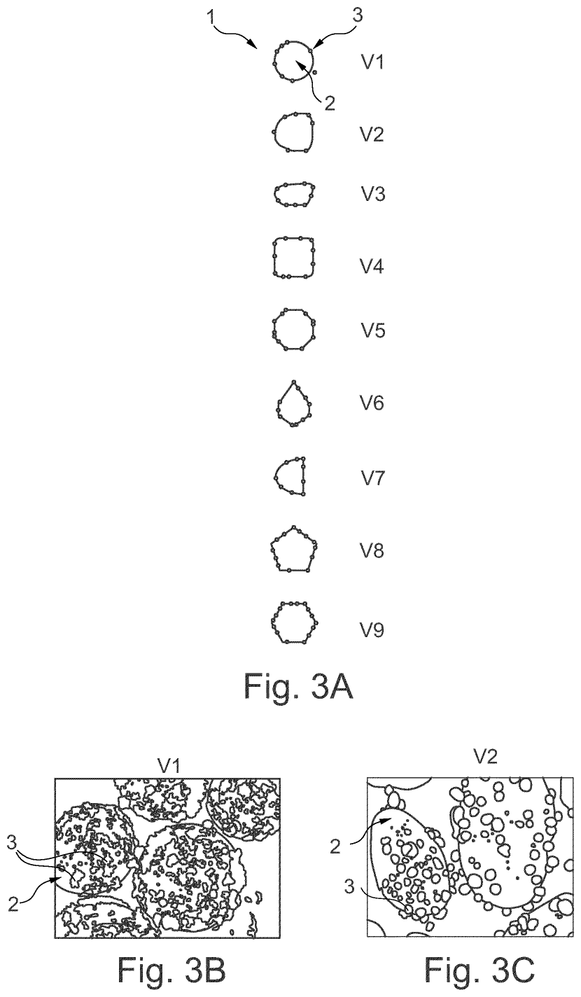

FIG. 3A shows conceivable particle shapes of the biogranule;

FIG. 3B shows a scanning electron microscope image of the implant 1 with round granular material particles;

FIG. 3C shows a scanning electron microscope image of the implant 1 with potato-shaped granular material particles;

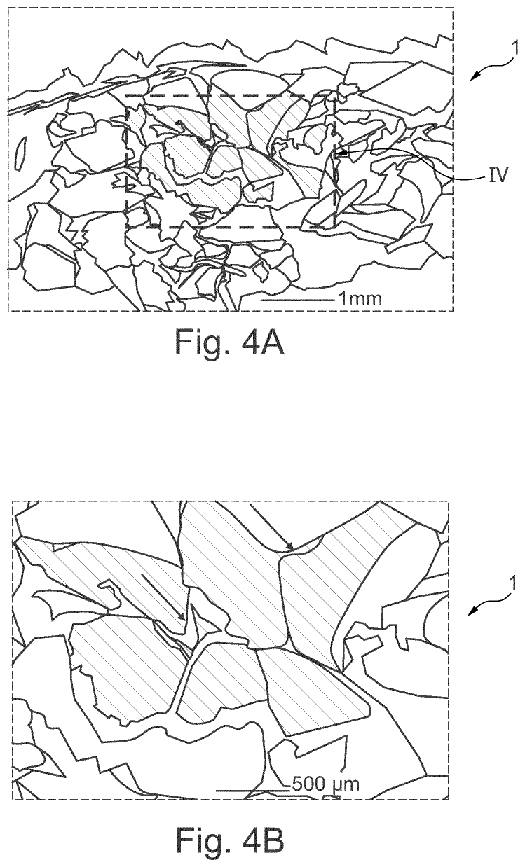

FIG. 4A shows a longitudinal sectional view of the implant 1 using a scanning electron microscope;

FIG. 4B shows the section IV from FIG. 4B.

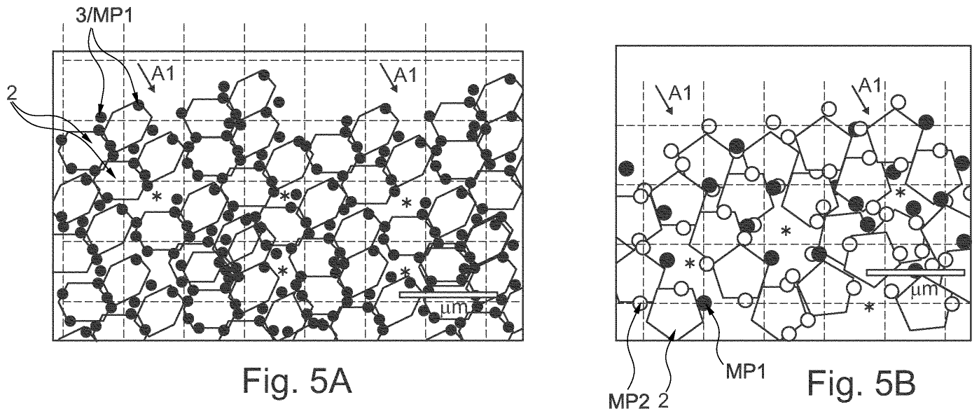

FIG. 5A shows a schematic representation of the implant 1 in the μm range with hexagonal granular material particles and a type of metal particles; and

FIG. 5B shows a schematic representation of the implant 1 in the μm range with pentagonal granular material particles and two types of metal particles.

The figures are only schematic in nature and serve only for the purpose of understanding the invention. The configuration example is purely exemplary.

FIG. 1 shows the implant 1, which comprises the base granular material 2 as well as the metal particles 3. It can be seen that both the base granular material 2 and the metal particles 3 are mixed together and are present in the implant 1 over the entire volume of the implant 1.

FIG. 2 shows a flow chart illustrating the steps of the method according to the invention. First, in the first step S1, a first raw material RM1, which is for example a biocompatible polymer (LDPE), and as a second raw material RM2 a ceramic granular material (for example calcium carbonate) are mixed together. By mixing these two raw materials, the base granular material 2 is obtained. In a second step S2, a first type of metal particles MP1, for example silver particles, and a second type of metal particles MP2, for example copper particles, are added to this base granular material 2 or are brought together with the base granular material 2 by blasting. After step S2, the implant mixture IM is obtained. In the third step S3 of the method, this implant mixture IM is pressed. This results in a material block which is crushed into chunks, for example by machining or grinding, which in turn are subsequently shaped into the final implant form. Thus, after step S3, the finished implant 1 is obtained, which can be placed/inserted into a patient body.

FIG. 3A shows, by way of example and without being limited thereto, nine different types/shapes/versions in which the particles of the biogranules 2 may be formed. Here, an implant 1 is assumed which has calcium carbonate as biogranules 2 and has, for example, silver particles, magnesium particles, etc. as metal particles 3. The particle types/particle shapes of the particles in the biogranules are continuously characterized by the symbols ‘V1’ to ‘V9’. The basic shape of the particles is round according to V1, potato-shaped according to V2, oval according to V3, square according to V4, octagon-shaped according to V5, parallelogon-shaped according to V6, semicircular according to V7, pentagon-shaped according to V8, and hexagon-shaped according to V9.

FIG. 3B shows a scanning electron microscope image of implant 1, which has round (V1) granular material particles in its biogranules 2. Here, UHMW-PE granular material is selected as biogranules 2 as an example. The metal particles 3 adhering to the entire surface of each individual granular material particle/biogranules 2 are silver particles here.

FIG. 3C shows, similarly to FIG. 3B, a scanning electron microscope image of the implant 1, which here has potato-shaped (V2) granular material particles. The implant 1 in FIG. 3C is composed of the same materials as the implant shown in FIG. 3B and differs from the latter only in the shape of its granular material particles 2.

FIG. 4A shows a longitudinal sectional view of the implant 1 using a scanning electron microscope. This is an example of a UHMW-PE implant with calcium carbonate particles mixed with magnesium particles, silver particles, etc. The implant 1 is porous in this case. Each particle of the granular material 2 has a layer of metal particles 3 distributed over its entire surface, which here stand out brightly against the granular material 2. Thus, the pore spaces (spaces between the individual particles of the granular material) are at least partially filled with metal particles 3.

FIG. 4B shows the section IV from FIG. 4A and thus the implant 1 from FIG. 4A on an enlarged scale.

FIG. 5A is a schematic representation of the implant 1 in the μm range, which here shows exemplary hexagonal/six-sided particles of biogranules 2, wherein UHMW-PE is chosen as biogranules 2 as an example. The dot-like/circle-like elements symbolize the metal particles 3 (of a metal type, for example MP1), which here may be silver, copper or zinc. The arrows A1 point in the direction of the porous surface of the implant 1. The ‘*’ symbol marks the areas between the granular material particles 2, i.e. the areas in the pores (pore spaces), which are characterized in particular by their intrinsically antimicrobially active pore structure.

Like FIG. 5A, FIG. 5B also shows a schematic representation of the implant 1 in the pm range. The two illustrations (FIG. 5A and FIG. 5B) of the implant 1 differ in that the granular material particles 2 in FIG. 5B are pentagonal/five-sided in shape and here, in addition to the metal particles 3 of type MP1, other particles MP2 with antimicrobial activity also adhere to these granular material particles 2, for example ceramic components, which are shown here as a polygon (regular decagon).

### LIST OF REFERENCE SIGNS

1 implant

2 base granular material

3 metal particles

IM implant mixture

RM1 raw material 1

RM2 raw material 2

MP1 metal particles 1

MP2 metal particles 2

S1 first step

S2 second step

S3 third step

V1 to V9 (nine different) variants of granular material shapes

1. An implant with antimicrobial activity comprising an implant mixture having a base granular material made of a raw material mixture of biocompatible polymers and/or a ceramic granular material, wherein the implant mixture further comprises at least one kind of particulate metal suitable for releasing ions, wherein the metal particles are provided in the form of silver particles and/or copper particles, wherein the metal particles are distributed in the volume of the implant so that the metal is interspersed with further metal particles in the form of magnesium particles and/or iron particles, which are highly pure and elemental as well as biodegradable metals.

2. The implant according claim 1, wherein the distribution, density, quantity and/or concentration of the metal particles in the implant mixture is such that the antimicrobial activity of the implant is forced to occur in its direct environment.

3. The implant according to claim 1, wherein the silver particles have a grain size in the range of 1-200 μm, the copper particles have a grain size in the range of 1-100 μm, and the magnesium particles and iron particles have a grain size in the range of 1-200 μm.

4. The implant according to claim 1, wherein the implant is porous in such a way that the antimicrobial activity of the porous implant is forced to occur on the pore surface.

5. The implant according to claim 1, wherein the implant is designed to be solid in such a way that the antimicrobial activity of the solid implant is forced to occur on the implant surface.

6. The implant according to claim 1, wherein the implant is produced with patient-specific shape and material properties.

7. The implant according to claim 1, wherein the implant is manufactured by compression molding, milling, laser sintering, or injection molding.

8. A method for producing an implant according to claim 1, characterized by the steps:
a) mixing of the raw materials for producing the base granular material;
b) mixing or blasting of the base granular material with the metal particles in a defined ratio, by which the implant mixture is formed, and
c) pressing the implant mixture for producing a material block which is crushed into chunks, and wherein these chunks are subsequently formed into the final implant shape.
